(19) United States
(12) Patent Application Publication
(10) Pub. No.: US 2025/0114830 A1
(43) Pub. Date: Apr. 10, 2025

(54) METHOD FOR RAPID AND HARMLESS TREATMENT OF LIVESTOCK AND POULTRY CARCASS/RESIDUE BY BACTERIUM-ENZYME COMPLEX

(71) Applicant: Xinjiang University, Urumqi (CN)

(72) Inventors: Haitao YUE, Urumqi (CN); Yanfei WANG, Urumqi (CN); Yiqian LUO, Urumqi (CN); Junqiang WU, Urumqi (CN); Ning WANG, Urumqi (CN); Zhengyang YANG, Urumqi (CN); Zhiyue LI, Urumqi (CN)

(73) Assignee: Xinjiang University, Urumqi (CN)

(21) Appl. No.: 18/483,030

(22) Filed: Oct. 9, 2023

(51) Int. Cl. B09B 3/60; C12N 1/20; C12N 9/20; C12N 9/50; C12R 1/01; C12R 1/07

(52) U.S. Cl. CPC B09B 3/60; C12N 1/205; C12N 9/20; C12N 9/50; C12Y 301/01001; C12Y 304/21; C12R 2001/01; C12R 2001/07

(57) ABSTRACT

The present disclosure provides a method for rapid and harmless treatment of a livestock and poultry carcass/residue by a bacterium-enzyme complex and relates to the technical field of dead livestock and poultry treatment. In the present disclosure, a composite microbial inoculant is compounded from protease-, lipase-, and keratinase-producing strains, and can be applied to the degradation of fats and proteins as well as deodorization. The composite microbial inoculant is further combined with a protease preparation and a lipase preparation to prepare the bacterium-enzyme complex. The bacterium-enzyme complex can quickly and harmlessly treat the carcasses/residues of pig, cattle, sheep, and chicken, and has a short treatment period, mild and controllable reaction conditions, and safe and non-toxic reaction process and reaction products. During the reaction, harmful gases such as CH4 and NH3 and greenhouse gases such as CO2 released by the method are less than those by other methods.

### TECHNICAL FIELD

The present disclosure belongs to the technical field of dead livestock and poultry treatment, and in particular relates to a method for rapid and harmless treatment of a livestock and poultry carcass/residue by a bacterium-enzyme complex.

### BACKGROUND

Breeding industry uses the physiological functions of domesticated animals such as livestock and poultry, or wild animals such as deer, musk deer, fox, mink, otter, and quail. The breeding industry converts plant energy including forage and feed into animal energy through artificial breeding and reproduction to obtain livestock and poultry products, such as meat, eggs, milk, wool, cashmere, hides, silk, and medicinal materials. The breeding industry in China mainly includes three sub-sectors of poultry farming, animal husbandry, and aquaculture, and the number of dead animals due to diseases shows a corresponding upward trend with an increase in the scale of industrial output.

In order to promote the healthy development of the livestock and poultry breeding industry and ensure the safety of the ecological environment, it is extremely necessary to do a good job in the harmless treatment of dead livestock and poultry caused by diseases. In China, conventional harmless treatment technologies for dead livestock and poultry caused by diseases currently mainly include 7 technologies from 4 processes, namely incineration, landfill, rendering, and fermentation. These technologies are all applied in the harmless treatment of dead livestock and poultry caused by diseases but show respective advantages and disadvantages. For the disadvantages, the incineration and rendering require special equipment with a relatively high cost; the landfill has the possibility of pathogenic microorganism contamination and occupies a large area; and the fermentation is limited by many factors, such as weather, venue, equipment, and artificial, and has a long-time consumption. Accordingly, it is necessary to improve the harmless treatment to reduce the amount of special equipment and save costs.

### SUMMARY

In view of this, an objective of the present disclosure is to provide a method for rapid and harmless treatment of a livestock and poultry carcass/residue by a bacterium-enzyme complex.

The method has a short reaction period as well as a safe and non-toxic reaction process and can obtain degradation products containing a large amount of fat and protein, thereby realizing resource utilization. Moreover, the method can also reduce the emission of greenhouse gases.

To achieve the above objective, the present disclosure provides the following technical solutions:

The present disclosure provides a composite microbial inoculant for treating a livestock and poultry carcass/residue, where the composite microbial inoculant includes Bacillus subtilis Bs XJU-1, Bacillus subtilis Bs XJU-2, Bacillus cereus Q2B1-2, Priestia megaterium Pm XJU-4, and Enterobacter hormaechei Eh XJU-5.

Preferably, the Bacillus subtilis Bs XJU-1, the Bacillus subtilis Bs XJU-2, the Bacillus cereus Q2B1-2, the Priestia megaterium Pm XJU-4, and the Enterobacter hormaechei Eh XJU-5 each have a viable count of not less than 108 cfu/g.

Preferably, the viable counts of the Bacillus subtilis Bs XJU-1, the Bacillus subtilis Bs XJU-2, the Bacillus cereus Q2B1-2, the Priestia megaterium Pm XJU-4, and the Enterobacter hormaechei Eh XJU-5 in the composite microbial inoculant are at a ratio of (1-5):(1-5):(1-5):(1-10):(1-5).

The present disclosure further provides a bacterium-enzyme complex for harmless treatment of a livestock and poultry carcass/residue, including a composite microbial inoculant, a protease preparation, and a lipase preparation that are packaged independently.

Preferably, the protease preparation includes an alkaline protease.

The present disclosure further provides a method for harmless treatment of a livestock and poultry carcass/residue using the bacterium-enzyme complex, including the following steps: (1) subjecting a livestock and poultry carcass/residue to be treated to crushing and a high-temperature high-pressure treatment at 100° C. to 120° C. and 0.10 MPa to 0.15 MPa;
(2) cooling a mixture I obtained in step (1), and mixing with the protease preparation to allow enzymolysis for 22 h to 26 h;
(3) mixing a mixture II obtained in step (2) with the lipase preparation to allow enzymolysis for 14 h to 18 h; and
(4) mixing a mixture III obtained in step (3) with the composite microbial inoculant to allow fermentative degradation for 22 h to 26 h.

Preferably, the mixture I in step (2) is cooled to 50° C. to 55° C. and then mixed with the protease preparation.

Preferably, the protease preparation in step (2) has a mass of 4% to 8% of a mass of the livestock and poultry carcass/residue.

Preferably, the mixture II in step (3) is cooled to 50° C. to 55° C. and then mixed with the lipase preparation; and the lipase preparation has a mass of 1% to 5% of a mass of the livestock and poultry carcass/residue.

Preferably, the mixture III in step (4) is cooled to 34° C. to 37° C. and then mixed with the composite microbial inoculant; and the composite microbial inoculant has a mass of 0.1% to 2% of a mass of the livestock and poultry carcass/residue.

Beneficial effects: the present disclosure provides a composite microbial inoculant for treating a livestock and poultry carcass/residue, where the composite microbial inoculant is compounded from protease-, lipase-, and keratinase-producing strains, and can be applied to the degradation of fats and proteins as well as deodorization. The composite microbial inoculant is further combined with a protease preparation and a lipase preparation to prepare the bacterium-enzyme complex. In the present disclosure, the bacterium-enzyme complex is used for rapid and harmless treatment of pig, chicken, cattle, and sheep carcasses/residues. (1) The method has a short treatment cycle of about 5 d, and shows small site occupation, which can be selected by different breeding enterprises according to their own breeding scale. (2) The method has mild and controllable reaction conditions. (3) The method has a safe and non-toxic reaction process and reaction products, and the reaction products contain a large amount of fatty acids and proteins, such that the reaction products can be used for resource utilization. (4) Compared with other methods, the method releases less methane and other gases during the reaction and is in line with the national policy of “reducing resource consumption and pollution discharge”.

### Deposit of Biological Material

Enterobacter hormaechei Eh XJU-5 was deposited in the China Center for Type Culture Collection (CCTCC) on Mar. 13, 2023 with a deposit number of CCTCC NO: M 2023306 (at the address: Wuhan University, Wuhan, China);

Priestia megaterium Pm XJU-4 was deposited in the CCTCC on Mar. 13, 2023 with a deposit number of CCTCC NO: M 2023309 (at the address: Wuhan University, Wuhan, China);

Bacillus subtilis Bs XJU-1 was deposited in the CCTCC on Mar. 13, 2023 with a deposit number of CCTCC NO: M 2023304 (at the address: Wuhan University, Wuhan, China);

Bacillus subtilis Bs XJU-2 was deposited in the CCTCC on Mar. 13, 2023 with a deposit number of CCTCC NO: M 2023305 (at the address: Wuhan University, Wuhan, China); and

Bacillus cereus Q2B1-2 was deposited in the China General Microbiological Culture Collection Center (CGMCC) on Jul. 25, 2022 with an deposit number of CGMCC NO. 25397 (at the address: Institute of Microbiology, Chinese Academy of Sciences, NO. 1 West Beichen Road, Chaoyang District, Beijing 100101).

### BRIEF DESCRIPTION OF THE DRAWINGS

FIG. 1 shows a degradation effect of the bacterium-enzyme complex on pork in the laboratory stage (front view).

FIG. 2 shows a degradation effect of the bacterium-enzyme complex on pork in the laboratory stage (top view).

FIG. 3 shows a degradation effect of the bacterium-enzyme complex on chicken in the laboratory stage (front view).

FIG. 4 shows a degradation effect of the bacterium-enzyme complex on chicken in the laboratory stage (top view).

FIG. 5 shows a degradation effect of the bacterium-enzyme complex on beef in the laboratory stage (front view).

FIG. 6 shows a degradation effect of the bacterium-enzyme complex on beef in the laboratory stage (top view).

FIG. 7 shows a degradation effect of the bacterium-enzyme complex on mutton in the laboratory stage (front view).

FIG. 8 shows a degradation effect of the bacterium-enzyme complex on mutton in the laboratory stage (top view).

FIG. 9 shows a degradation effect of the bacterium-enzyme complex on meat mixture in the laboratory stage (front view).

FIG. 10 shows a degradation effect of the bacterium-enzyme complex on meat mixture in the laboratory stage (top view).

FIG. 11 shows a degradation effect of the bacterium-enzyme complex on pork in a pilot test stage of a 3-ton fermentor.

FIG. 12 shows gas production during the process of degradation of livestock and poultry residues by the bacterium-enzyme complex.

### DETAILED DESCRIPTION OF THE EMBODIMENTS

The present disclosure provides a composite microbial inoculant for treating a livestock and poultry carcass/residue, where the composite microbial inoculant includes Bacillus subtilis Bs XJU-1, Bacillus subtilis Bs XJU-2, Bacillus cereus Q2B1-2, Priestia megaterium Pm XJU-4, and Enterobacter hormaechei Eh XJU-5.

In the present disclosure, the viable counts of the Bacillus subtilis Bs XJU-1, the Bacillus subtilis Bs XJU-2, the Bacillus cereus Q2B1-2, the Priestia megaterium Pm XJU-4, and the Enterobacter hormaechei Eh XJU-5 in the composite microbial inoculant are at a ratio of (1-5):(1-5):(1-5):(1-10):(1-5). The above strains are preferably isolated and selected from saline-alkali land in Xinjiang. These strains Bacillus subtilis Bs XJU-1, Bacillus subtilis Bs XJU-2, Bacillus cereus Q2B1-2, Priestia megaterium Pm XJU-4, and Enterobacter hormaechei Eh XJU-5 show high temperature resistance, strong adhesion, gastric acid resistance, and bile salt resistance, are safe, non-toxic, and drug residue-free, and can be used to degrade livestock and poultry carcasses (residues). The Bacillus subtilis Bs XJU-1, Bacillus subtilis Bs XJU-2, and Priestia megaterium Pm XJU-4 have an ability to secrete protease and antibacterial substances and can be used to degrade the main component protein of livestock and poultry carcasses (residues). The Bacillus subtilis Bs XJU-1, Bacillus subtilis Bs XJU-2, and Priestia megaterium Pm XJU-4 also have an ability to produce lipase, and can be used for the degradation of fat, the main component of animal carcasses (residues). The Enterobacter hormaechei Eh XJU-5 has an ability to produce keratinase and deodorize and can be used for the degradation of dead poultry feathers and deodorization.

The present disclosure further provides a bacterium-enzyme complex for harmless treatment of a livestock and poultry carcass/residue, including a composite microbial inoculant, a protease preparation, and a lipase preparation that are packaged independently.

In the present disclosure, the composite microbial inoculant includes the Bacillus subtilis Bs XJU-1, the Bacillus subtilis Bs XJU-2, the Bacillus cereus Q2B1-2, the Priestia megaterium Pm XJU-4, and the Enterobacter hormaechei Eh XJU-5. The protease preparation includes preferably conventional commercially available proteases. As in an example, an alkaline protease, a neutral protease, and papain are mixed according to a mass ratio of (2-5):(3-4):(1-3), but it cannot only be regarded as the entire protection scope of the present disclosure. There is no special limitation on a source of the protease, and conventional commercially available products are preferred. The lipase preparation includes one or a combination of more lipases. As in an example, an acidic lipase and a neutral lipase are mixed in a mass ratio of (1-4):(2-4), but it cannot only be considered as the entire protection scope of the present disclosure. There is no special limitation on a source of the lipase, and conventional commercially available products are preferred.

In the present disclosure, the composite microbial inoculant is preferably used to degrade proteins and fat obtained by enzymatic hydrolysis of protease and lipase; and the viable counts of the Bacillus subtilis Bs XJU-1, the Bacillus subtilis Bs XJU-2, the Bacillus cereus Q2B1-2, the Priestia megaterium Pm XJU-4, and the Enterobacter hormaechei Eh XJU-5 are at a ratio of preferably (1-5):(1-5):(1-5):(1-10):(1-5), more preferably (1-2):(1-3):(1-2):(2-5):(2-4). A preparation method of the composite microbial inoculant preferably includes: mixing and culturing the strains in the composite microbial inoculant in an LB medium until reaching an end of a logarithmic growth phase to an early stage of a plateau phase, and conducting freeze-drying to obtain a bacterial powder.

The present disclosure further provides a method for harmless treatment of a livestock and poultry carcass/residue using the bacterium-enzyme complex, including the following steps: (1) subjecting a livestock and poultry carcass/residue to be treated to crushing and a high-temperature high-pressure treatment at 100° C. to 120° C. and 0.12 MPa to 0.15 MPa;
(2) cooling a mixture I obtained in step (1), and mixing with the protease preparation to allow enzymolysis for 22 h to 26 h;
(3) mixing a mixture II obtained in step (2) with the lipase preparation to allow enzymolysis for 14 h to 18 h; and
(4) mixing a mixture III obtained in step (3) with the composite microbial inoculant to allow fermentative degradation for 22 h to 26 h.

In the present disclosure, a livestock and poultry carcass/residue to be treated is subjected to crushing and a high-temperature high-pressure treatment at 100° C. to 120° C. and 0.12 MPa to 0.15 MPa. The crushing preferably includes crushing to obtain a particle size of 5 cm×5 cm×5 cm. When the high-temperature high-pressure treatment is conducted, pulverized meat particles and water are preferably mixed according to a volume ratio of 1:(1-2), and then the high-temperature high-pressure treatment is conducted for 1 h to 2 h. The high-temperature high-pressure treatment can kill microorganisms in the environment and the animal carcass itself to prevent contamination.

In the present disclosure, a mixture I obtained is cooled, and mixed with the protease preparation to allow enzymolysis for 22 h to 26 h. The mixture I is preferably cooled to 50° C. to 55° C. and then mixed with the protease preparation; and the protease preparation has a mass of preferably 4% to 8%, more preferably 5.2%, of a mass of the pig, chicken, cattle, and sheep carcass/residue. The proteins in pork are enzymatically hydrolyzed with the protease preparation.

In the present disclosure, a mixture II obtained is mixed with the lipase preparation to allow enzymolysis for 14 h to 18 h. The mixture II is preferably cooled to 50° C. to 55° C. and then with the lipase preparation; and the lipase preparation has a mass of preferably 1% to 5%, more preferably 1.7%, of a mass of the pig, chicken, cattle, and sheep carcass/residue. The fats in pork are enzymatically hydrolyzed with the lipase preparation.

In the present disclosure, a mixture III obtained is mixed with the composite microbial inoculant to allow fermentative degradation for 22 h to 26 h. The mixture III is preferably cooled to 34° C. to 37° C. and then with the composite microbial inoculant; and the composite microbial inoculant has a mass of preferably 0.1% to 2%, more preferably 0.3%, of a mass of the pig, chicken, cattle, and sheep carcass/residue. The fermentative degradation preferably includes facultative anaerobic fermentation. The composite microbial inoculant continues to degrade a product obtained after enzymolysis. When the composite microbial inoculant is added, glucose with a total weight of 1% of the enzymolysis solution is further preferably added and stirred evenly.

In the present disclosure, after the fermentative degradation is completed, a treated mixture is preferably heated to 70° C. and held for 3 h to allow inactivation and canned, and a resulting fermented stock solution is preserved by canning for subsequent resource reuse.

The method for rapid and harmless treatment of a livestock and poultry carcass/residue by a bacterium-enzyme complex provided by the present disclosure will be described in detail below in conjunction with the examples, but they cannot be interpreted as limiting the protection scope of the present disclosure. In addition, in the examples of the present disclosure, the degradation can be conducted within the following ranges, and a time and amounts of bacterial powder and enzyme powder can be increased or decreased according to the amount of animal carcasses.

### Example 1

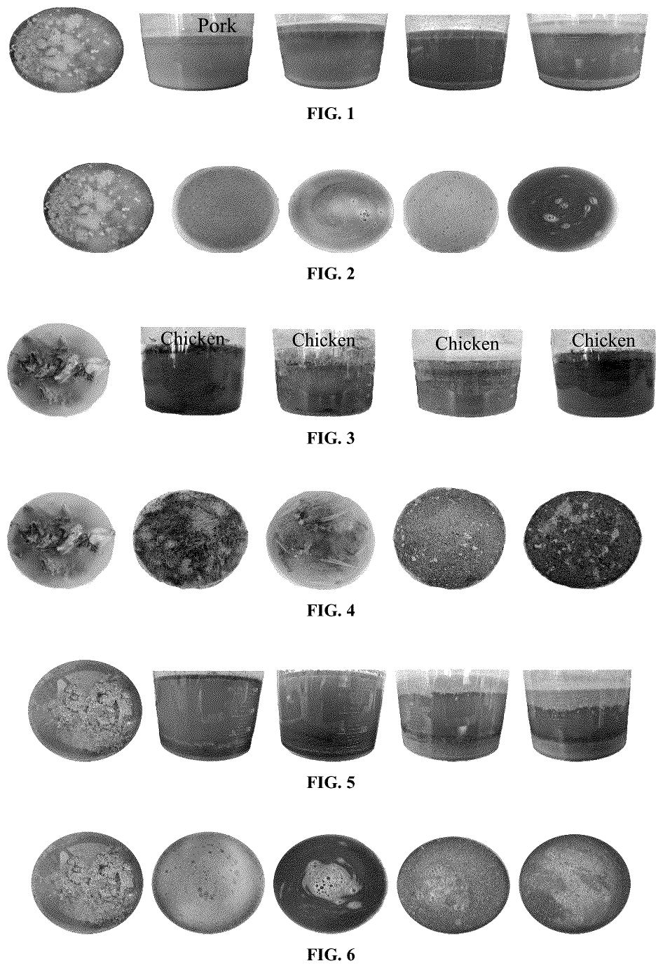

Under laboratory conditions, 1 kg of pork was subjected to harmless treatment with a bacterium-enzyme complex. The treatment included the following steps:
1) the pork was crushed, water was added according to a volume ratio of the carcass and water at 1:2, and high-temperature high-pressure treatment was conducted at 115° C. and 0.12 MPa for 1 h;
2) a mixture I obtained in step 1) was cooled to 55° C., added with a protease preparation (1) (at a dosage of 5.2%, and including an alkaline protease:a neutral protease:papain=2:3:1), and stirred evenly to allow a reaction for 24 h;
3) a mixture II obtained in step 2) was kept at 50° C., added with a lipase preparation (2) (at a dosage of 1.7%, and including an acidic lipase:a neutral lipase=1:2), and stirred evenly to allow a reaction for 16 h;
4) a mixture III obtained in step 3) was cooled to 37° C., added with a composite microbial inoculant serving as an enzymolysis solution (at a dosage of 0.3%, and including Bacillus subtilis Bs XJU-1, Bacillus subtilis Bs XJU-2, Bacillus cereus Q2B1-2, Priestia megaterium Pm XJU-4, and Enterobacter hormaechei Eh XJU-5, with a viable count ratio of (1-2):(1-3):(1-2):(2-5):(2-4)), added with glucose at 1% of a total weight of the enzymolysis solution, and stirred evenly to allow fermentation for 24 h; and
5) a mixture IV obtained in step 4) was heated to 70° C. and held for 3 h to allow inactivation, and then canned. The final degradation results were shown in FIG. 1 (front view) and FIG. 2 (top view).

### Example 2

Under laboratory conditions, 1 kg of a chicken residue was subjected to harmless treatment with a bacterium-enzyme complex. The treatment included the following steps:
1) the chicken residue was crushed, water was added according to a volume ratio of the carcass and water at 1:2, and high-temperature high-pressure treatment was conducted at 120° C. and 0.12 MPa for 1 h;
2) a mixture I obtained in step 1) was cooled to 50° C., added with a protease preparation (1) (at a dosage of 5.2%, and including an alkaline protease:a neutral protease:papain=2:3:1), and stirred evenly to allow a reaction for 22 h;
3) a mixture II obtained in step 2) was kept at 50° C., added with a lipase preparation (2) (at a dosage of 1.7%, and including an acidic lipase:a neutral lipase=1:2), and stirred evenly to allow a reaction for 14 h;
4) a mixture III obtained in step 3) was cooled to 35° C., added with a composite microbial inoculant serving as an enzymolysis solution (at a dosage of 0.3%), added with glucose at 1% of a total weight of the enzymolysis solution, and stirred evenly to allow fermentation for 22 h; and
5) a mixture IV obtained in step 4) was heated to 70° C. and held for 3 h to allow inactivation, and then canned. The final degradation results were shown in FIG. 3 (front view) and FIG. 4 (top view).

### Example 3

Under laboratory conditions, 1 kg of beef was subjected to harmless treatment with a bacterium-enzyme complex. The treatment included the following steps:
1) the beef was crushed, water was added according to a volume ratio of the carcass and water at 1:2, and high-temperature high-pressure treatment was conducted at 112° C. and 0.12 MPa for 1 h;
2) a mixture I obtained in step 1) was cooled to 50° C., added with a protease preparation (1) (at a dosage of 5.2%, and including an alkaline protease:a neutral protease:papain=2:3:1), and stirred evenly to allow a reaction for 24 h;
3) a mixture II obtained in step 2) was kept at 52° C., added with a lipase preparation (2) (at a dosage of 1.7%, and including an acidic lipase:a neutral lipase=(1-4):(2-4)), and stirred evenly to allow a reaction for 15 h;
4) a mixture III obtained in step 3) was cooled to 34° C., added with a composite microbial inoculant serving as an enzymolysis solution (at a dosage of 0.3%), added with glucose at 1% of a total weight of the enzymolysis solution, and stirred evenly to allow fermentation for 22 h; and
5) a mixture IV obtained in step 4) was heated to 70° C. and held for 3 h to allow inactivation, and then canned. The final degradation results were shown in FIG. 5 (front view) and FIG. 6 (top view).

### Example 4

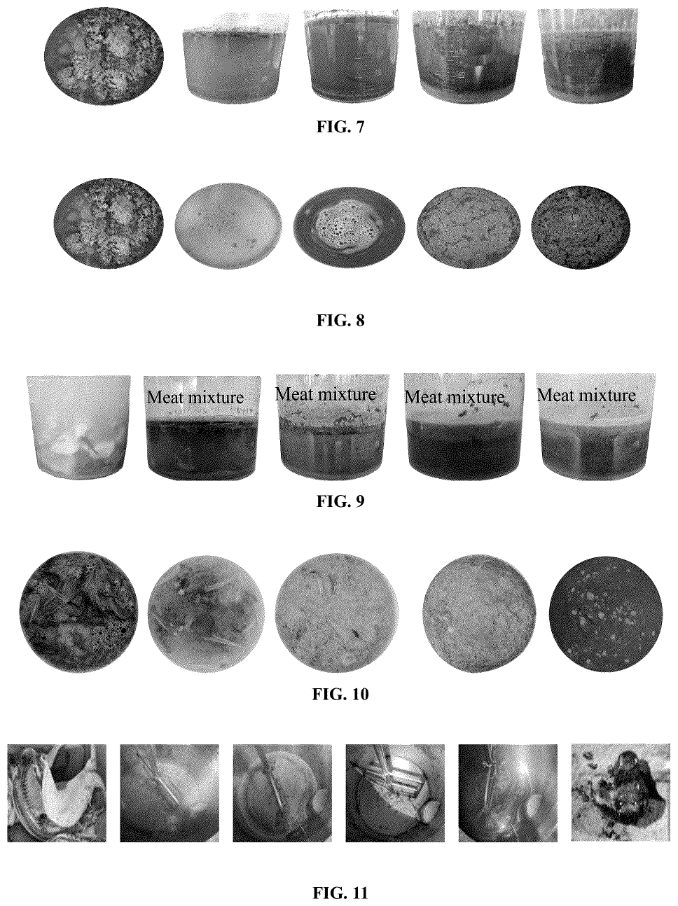

Under laboratory conditions, 1 kg of mutton was subjected to harmless treatment with a bacterium-enzyme complex. The treatment included the following steps:
1) the mutton was crushed, water was added according to a volume ratio of the carcass and water at 1:2, and high-temperature high-pressure treatment was conducted at 110° C. and 0.13 MPa for 1 h;
2) a mixture I obtained in step 1) was cooled to 52° C., added with a protease preparation (1) (at a dosage of 5.2%, and including an alkaline protease:a neutral protease:papain=(2-5):(3-4):(1-3)), and stirred evenly to allow a reaction for 25 h;
3) a mixture II obtained in step 2) was kept at 55° C., added with a lipase preparation (2) (at a dosage of 1.7%, and including an acidic lipase:a neutral lipase=(1-4):(2-4)), and stirred evenly to allow a reaction for 18 h;
4) a mixture III obtained in step 3) was cooled to 37° C., added with a composite microbial inoculant serving as an enzymolysis solution (at a dosage of 0.3%), added with glucose at 1% of a total weight of the enzymolysis solution, and stirred evenly to allow fermentation for 25 h; and
5) a mixture IV obtained in step 4) was heated to 70° C. and held for 3 h to allow inactivation, and then canned. The final degradation results were shown in FIG. 7 (front view) and FIG. 8 (top view).

### Example 5

1 kg each of pork, beef, mutton, and chicken was used as a substrate for the rapid degradation of a bacterium-enzyme complex. The treatment included the following steps:
1) a meat mixture was crushed, water was added according to a volume ratio of the carcass and water at 1:2, and high-temperature high-pressure treatment was conducted at 115° C. and 0.12 MPa for 1.5 h;
2) a mixture I obtained in step 1) was cooled to 55° C., added with a protease preparation (1) (at a dosage of 2 wt % of the meat mixture, and including an alkaline protease:a neutral protease=2:1) and stirred evenly to allow a reaction for 24 h;
3) a mixture II obtained in step 2) was kept at 50° C., added with a lipase preparation (at a dosage of 2.0 wt % of the meat mixture, and including an acidic lipase:a neutral lipase=1:2), and stirred evenly to allow a reaction for 18 h;
4) a mixture III obtained in step 3) was cooled to 37° C., added with a composite microbial inoculant serving as an enzymolysis solution (at a dosage of 5 wt %), added with glucose at 1% of a total weight of the enzymolysis solution, and stirred evenly to allow enzyme-compounded degradation by facultative anaerobic bacteria for 22 h; and
5) a mixture IV obtained in step 4) was heated to 60° C. and held for 4 h to allow inactivation to complete the degradation. The final degradation results were shown in FIG. 9 and FIG. 10.

### Example 6

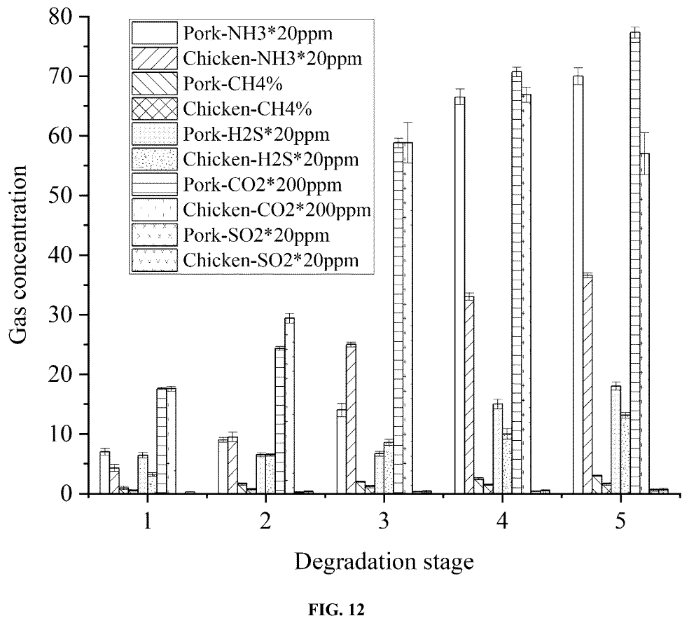

In a 3-ton fermentor, 100 kg of pork was subjected to harmless treatment with a bacterium-enzyme complex. The treatment included the following steps:
1) the pork was crushed, water was added according to a volume ratio of the carcass and water at 1:2, and high-temperature high-pressure treatment was conducted at 120° C. and 0.13 MPa for 1 h;
2) a mixture I obtained in step 1) was cooled to 56° C., added with a protease preparation (1) (at a dosage of 5.2%, and including an alkaline protease:a neutral protease:papain=(2-5):(3-4):(1-3)), and stirred evenly to allow a reaction for 24 h;
3) a mixture II obtained in step 2) was kept at 53° C., added with a lipase preparation (2) (at a dosage of 1.7%, and including an acidic lipase:a neutral lipase=(1-4):(2-4)), and stirred evenly to allow a reaction for 18 h;
4) a mixture III obtained in step 3) was cooled to 36° C., added with a composite microbial inoculant serving as an enzymolysis solution (at a dosage of 0.5%), added with glucose at 1% of a total weight of the enzymolysis solution, and stirred evenly to allow fermentation for 25 h; and
5) a mixture IV obtained in step 4) was heated to 70° C. and held for 3 h to allow inactivation, and then canned. The final degradation results were shown in FIG. 11. A scale-up experiment was conducted on the basis of laboratory conditions, and it was seen from the figure that the pork was completely degraded, indicating that this method could be applied to actual work. The results of gas released during the treatment of livestock and poultry carcasses/residues with the bacterium-enzyme complex were shown in FIG. 12.

The above descriptions are merely preferred implementations of the present disclosure. It should be noted that a person of ordinary skill in the art may further make several improvements and modifications without departing from the principle of the present disclosure, and such improvements and modifications should be deemed as falling within the protection scope of the present disclosure.

What is claimed is:

1. A composite microbial inoculant for treating a livestock and poultry carcass/residue, wherein the composite microbial inoculant comprises Bacillus subtilis Bs XJU-1, Bacillus subtilis Bs XJU-2, Bacillus cereus Q2B1-2, Priestia megaterium Pm XJU-4, and Enterobacter hormaechei Eh XJU-5.

2. The composite microbial inoculant according to claim 1, wherein the Bacillus subtilis Bs XJU-1, the Bacillus subtilis Bs XJU-2, the Bacillus cereus Q2B1-2, the Priestia megaterium Pm XJU-4, and the Enterobacter hormaechei Eh XJU-5 each have a viable count of not less than 108 cfu/g.

3. The composite microbial inoculant according to claim 1, wherein the viable counts of the Bacillus subtilis Bs XJU-1, the Bacillus subtilis Bs XJU-2, the Bacillus cereus Q2B1-2, the Priestia megaterium Pm XJU-4, and the Enterobacter hormaechei Eh XJU-5 are at a ratio of (1-5):(1-5):(1-5):(1-10):(1-5).

4. The composite microbial inoculant according to claim 2, wherein the viable counts of the Bacillus subtilis Bs XJU-1, the Bacillus subtilis Bs XJU-2, the Bacillus cereus Q2B1-2, the Priestia megaterium Pm XJU-4, and the Enterobacter hormaechei Eh XJU-5 are at a ratio of (1-5):(1-5):(1-5):(1-10):(1-5).

5. A bacterium-enzyme complex for harmless treatment of a livestock and poultry carcass/residue, comprising a composite microbial inoculant, a protease preparation, and a lipase preparation that are packaged independently.

6. The bacterium-enzyme complex according to claim 5, wherein the protease preparation comprises an alkaline protease.

7. A method for harmless treatment of a livestock and poultry carcass/residue using the bacterium-enzyme complex according to claim 5, comprising the following steps:
(1) subjecting a livestock and poultry carcass/residue to be treated to crushing and a high-temperature high-pressure treatment at 100° C. to 120° C. and 0.10 MPa to 0.15 MPa;
(2) cooling a mixture I obtained in step (1), and mixing with the protease preparation to allow enzymolysis for 22 h to 26 h;
(3) mixing a mixture II obtained in step (2) with the lipase preparation to allow enzymolysis for 14 h to 18 h; and
(4) mixing a mixture III obtained in step (3) with the composite microbial inoculant to allow fermentative degradation for 22 h to 26 h.

8. The method according to claim 7, wherein the protease preparation comprises an alkaline protease.

9. The method according to claim 7, wherein the mixture I is cooled to 50° C. to 55° C. and then mixed with the protease preparation in step (2).

10. The method according to claim 8, wherein the mixture I is cooled to 50° C. to 55° C. and then mixed with the protease preparation in step (2).

11. The method according to claim 7, wherein the protease preparation in step (2) has a mass of 4% to 8% of a mass of the livestock and poultry carcass/residue.

12. The method according to claim 8, wherein the protease preparation in step (2) has a mass of 4% to 8% of a mass of the livestock and poultry carcass/residue.

13. The method according to claim 9, wherein the protease preparation in step (2) has a mass of 4% to 8% of a mass of the livestock and poultry carcass/residue.

14. The method according to claim 10, wherein the protease preparation in step (2) has a mass of 4% to 8% of a mass of the livestock and poultry carcass/residue.

15. The method according to claim 9, wherein the mixture II is cooled to 50° C. to 55° C. and then mixed with the lipase preparation in step (3); and the lipase preparation has a mass of 1% to 5% of a mass of the livestock and poultry carcass/residue.

16. The method according to claim 10, wherein the mixture II is cooled to 50° C. to 55° C. and then mixed with the lipase preparation in step (3); and the lipase preparation has a mass of 1% to 5% of a mass of the livestock and poultry carcass/residue.

17. The method according to claim 9, wherein the mixture III is cooled to 34° C. to 37° C. and then mixed with the composite microbial inoculant in step (4); and the composite microbial inoculant has a mass of 0.1% to 2% of a mass of the livestock and poultry carcass/residue.

18. The method according to claim 10, wherein the mixture III is cooled to 34° C. to 37° C. and then mixed with the composite microbial inoculant in step (4); and the composite microbial inoculant has a mass of 0.1% to 2% of a mass of the livestock and poultry carcass/residue.
